(19) United States
(12) Patent Application Publication
(10) Pub. No.: US 2023/0137803 A1
(43) Pub. Date: May 4, 2023

(54) JAW MEMBER, SURGICAL INSTRUMENT INCLUDING AT LEAST ONE JAW MEMBER, AND METHOD OF MANUFACTURING A JAW MEMBER

(71) Applicant: Covidien LP, Mansfield, MA (US)

(72) Inventors: Michael B. Lyons, Boulder, CO (US); Matthew S. Cowley, Frederick, CO (US); David J. Van Tol, Boulder, CO (US); William E. Robinson, Boulder, CO (US)

(21) Appl. No.: 17/954,142

(22) Filed: Sep. 27, 2022

Related U.S. Application Data
(60) Provisional application No. 63/274,886, filed on Nov. 2, 2021.

(51) Int. Cl. A61B 17/28; A61B 17/29; B21J 5/02

(52) U.S. Cl. CPC A61B 17/282; A61B 17/29; B21J 5/02; A61B 2017/00526

(57) ABSTRACT

A jaw member of a surgical instrument includes a structural body including a tissue-facing surface having an elongated opening defined therethrough and defining an internal cavity in communication with the elongated opening. A jaw liner of a material that is not conducive to molding includes an internal portion substantially filling the internal cavity and extending to the elongated opening. A method of manufacturing a jaw member of a surgical instrument includes preparing a material, forging the material into an internal cavity defined within a structural body of a jaw member, and setting the material such that the material substantially fills the internal cavity and is retained therein thereby forming a jaw liner of the jaw member.

### CROSS-REFERENCE TO RELATED APPLICATIONS

This application claims the benefit of, and priority to, U.S. Provisional Patent Application No. 63/274,886, filed on Nov. 2, 2021, the entire contents of which are hereby incorporated herein by reference.

### FIELD

The present disclosure relates to surgical instruments and, more particularly, to jaw members, surgical instruments including at least one jaw member, and methods of manufacturing jaw members of surgical instruments.

### BACKGROUND

Ultrasonic surgical instruments and systems utilize ultrasonic energy, i.e., ultrasonic vibrations, to treat tissue. More specifically, ultrasonic surgical instruments and systems utilize mechanical vibration energy transmitted at ultrasonic frequencies to treat tissue. An ultrasonic surgical device may include, for example, an ultrasonic blade and a clamp mechanism to enable clamping of tissue against the blade. Ultrasonic energy transmitted to the blade causes the blade to vibrate at very high frequencies, which allows for heating tissue to treat tissue clamped against or otherwise in contact with the blade.

Electrosurgical instruments and systems conduct Radio Frequency (RF) energy through tissue to treat tissue. An electrosurgical instrument or system may be configured to conduct bipolar RF energy between oppositely charged electrodes and through tissue, e.g., tissue clamped between the electrodes or otherwise in contact therewith, to treat tissue. Alternatively or additionally, an electrosurgical instrument or system may be configured to deliver monopolar RF energy from an active electrode to tissue in contact with the electrode, with the energy returning via a remote return electrode device to complete the circuit.

### SUMMARY

As used herein, the term “distal” refers to the portion that is described which is further from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is a jaw member of a surgical instrument including a structural body and a jaw liner. The structural body includes a tissue-facing surface having an elongated opening defined therethrough and defines an internal cavity in communication with the elongated opening. The jaw liner material is not conducive to molding and is more malleable and plastically deformable through heat and/or pressure, while still maintaining its structural integrity, such that, under heat and/or pressure, the jaw liner can be formed into a desired shape. The jaw liner includes an internal portion substantially filling the internal cavity and extending to the elongated opening.

In an aspect of the present disclosure, the material is PTFE.

In another aspect of the present disclosure, the jaw liner includes an external portion that extends from the elongated opening and protrudes from the tissue-facing surface and/or extends outwardly over the tissue-facing surface.

In yet another aspect of the present disclosure, the external portion of the jaw liner defines at least one feature therein or thereon.

In still another aspect of the present disclosure, the internal cavity defines first and second portions having different widths. The first portion is disposed in direct communication with the elongated opening and the second portion communicates with the elongated opening through the first portion.

In still yet another aspect of the present disclosure, the structural body includes at least one retention feature extending into the internal cavity and configured to facilitate retention of the jaw liner.

In another aspect of the present disclosure, the structural body includes a raised mesa extending from the tissue-facing surface. The elongated opening is defined through the raised mesa. In such aspects, the jaw liner may include an external portion that extends from the elongated opening outwardly over at least a portion of the raised mesa.

In another aspect of the present disclosure, except for the elongated opening, the structural body substantially encloses the internal cavity.

A method of manufacturing a jaw member of a surgical instrument provided in accordance with the present disclosure includes preparing a material, forging the material into an internal cavity defined within a structural body of a jaw member, and setting the material once the material substantially fills the internal cavity and is retained therein thereby forming a jaw liner of the jaw member.

In an aspect of the present disclosure, prior to forging the material, the method includes positioning the structural body within a fixture.

In another aspect of the present disclosure, the forging includes impression die forging.

In another aspect of the present disclosure, preparing the material includes heating the material.

In still another aspect of the present disclosure, the material is PTFE or another material that cannot be molded (e.g., that does not flow in a manner conducive to plastic molding when heated above a melting point thereof).

In yet another aspect of the present disclosure, forging the prepared material includes urging the material through an elongated opening defined within a tissue-facing surface of the structural body and into the internal cavity.

In still yet another aspect of the present disclosure, setting the material includes actively cooling the material or allowing the material to cool.

In another aspect of the present disclosure, forging the material into the internal cavity defined within the structural body further includes forging a portion of the material externally of the structural body. Forging the portion of the material externally of the structural body may include protruding the portion of material from or extending the portion of material along the tissue-facing surface of the structural body.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 is a side view of a surgical system provided in accordance with the present disclosure including a surgical instrument, a surgical generator, and a return electrode device;

FIG. 2 is perspective view of another surgical system provided in accordance with the present disclosure including a surgical instrument incorporating an ultrasonic generator, electrosurgical generator, and power source therein;

FIG. 3 is a schematic illustration of a robotic surgical system provided in accordance with the present disclosure;

FIG. 4 is a longitudinal, cross-sectional view of a distal end portion of the surgical instrument of FIG. 1;

FIG. 5 is a transverse, cross-sectional view of the end effector assembly of the surgical instrument of FIG. 1;

FIG. 6 is a transverse, cross-sectional view of another configuration of the end effector assembly of the surgical instrument of FIG. 1;

FIGS. 7A and 7B are perspective and sectioned perspective views, respectively, of a portion of a jaw member provided in accordance with the present disclosure and configured for use with the surgical instrument of FIG. 1, the surgical instrument of FIG. 2, or any other suitable surgical instrument;

FIG. 8 is a perspective view of the portion of the jaw member of FIGS. 7A and 7B without the jaw liner;

FIG. 9 is a perspective view of the portion of the jaw member of FIGS. 7A and 7B without the structural body;

FIGS. 10A and 10B are transverse, cross-sectional views of another jaw member provided in accordance with the present disclosure and configured for use with the surgical instrument of FIG. 1, the surgical instrument of FIG. 2, or any other suitable surgical instrument, without and with the jaw liner, respectively;

FIG. 11 is a sectioned perspective view of another jaw member provided in accordance with the present disclosure and configured for use with the surgical instrument of FIG. 1, the surgical instrument of FIG. 2, or any other suitable surgical instrument, without the jaw liner;

FIG. 12 is a flow diagram illustrating a method of manufacturing a jaw member in accordance with the present disclosure.

### DETAILED DESCRIPTION

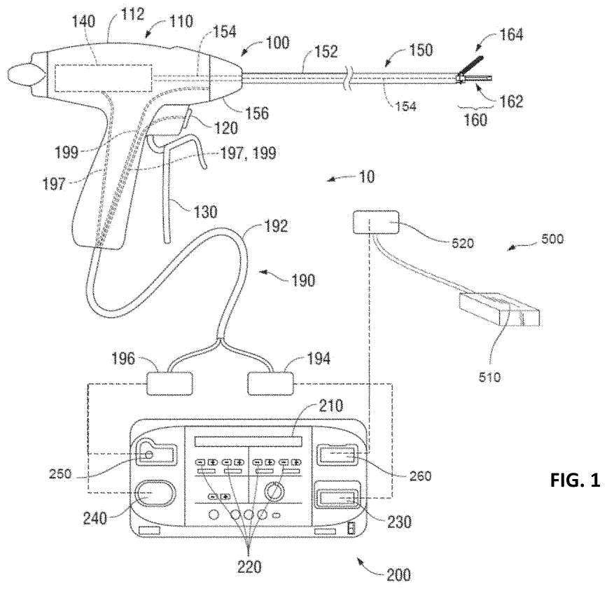

Referring to FIG. 1, a surgical system provided in accordance with aspects of the present disclosure is shown generally identified by reference numeral 10 including a surgical instrument 100, a surgical generator 200, and, in some aspects, a return electrode device 500, e.g., including a return pad 510. Surgical instrument 100 includes a handle assembly 110, an elongated assembly 150 extending distally from handle assembly 110, an end effector assembly 160 disposed at a distal end of elongated assembly 150, and a cable assembly 190 operably coupled with handle assembly 110 and extending therefrom for connection to surgical generator 200.

Surgical generator 200 includes a display 210, a plurality user interface features 220, e.g., buttons, touch screens, switches, etc., an ultrasonic plug port 230, a bipolar electrosurgical plug port 240, and active and return monopolar electrosurgical plug ports 250, 260, respectively. As an alternative to plural dedicated ports 230-260, one or more common ports (not shown) may be configured to act as any two or more of ports 230-260.

Surgical instrument 100 may be configured to operate in one or more electrosurgical modes supplying Radio Frequency (RF) energy to tissue to treat tissue, e.g., a monopolar configuration and/or a bipolar configuration, and/or in an ultrasonic mode supplying ultrasonic energy to tissue to treat tissue. Other additional or alternative energy modalities are also contemplated such as, for example, microwave energy, thermal energy, light energy, etc. Surgical generator 200 is configured to produce ultrasonic drive signals for output through ultrasonic plug port 230 to surgical instrument 100 to activate surgical instrument 100 in the ultrasonic mode (where so provided) and/or to provide electrosurgical energy, e.g., RF bipolar energy for output through bipolar electrosurgical plug port 240 and/or RF monopolar energy for output through active monopolar electrosurgical port 250 to surgical instrument 100 to activate surgical instrument 100 in the one or more electrosurgical modes (where so provided). Plug 520 of return electrode device 500 is configured to connect to return monopolar electrosurgical plug port 260 to return monopolar electrosurgical energy from surgical instrument 100 in the monopolar electrosurgical mode.

Continuing with reference to FIG. 1, handle assembly 110 includes a housing 112, an activation button 120, and a clamp trigger 130. Housing 112 is configured to support an ultrasonic transducer 140. Ultrasonic transducer 140 may be permanently engaged within housing 112 or removable therefrom. Ultrasonic transducer 140 includes a piezoelectric stack other suitable ultrasonic transducer components electrically coupled to surgical generator 200, e.g., via one or more of first electrical lead wires 197, to enable communication of ultrasonic drive signals to ultrasonic transducer 140 to drive ultrasonic transducer 140 to produce ultrasonic vibration energy that is transmitted along a waveguide 154 of elongated assembly 150 to blade 162 of end effector assembly 160 of elongated assembly 150, as detailed below. Feedback and/or control signals may likewise be communicated between ultrasonic transducer 140 and surgical generator 200. Ultrasonic transducer 140, more specifically, may include a stack of piezoelectric elements secured, under pre-compression between proximal and distal end masses or a proximal end mass and an ultrasonic horn with first and second electrodes electrically coupled between piezoelectric elements of the stack of piezoelectric elements to enable energization thereof to produce ultrasonic energy. However, other suitable ultrasonic transducer configurations, including plural transducers and/or non-longitudinal, e.g., torsional, transducers are also contemplated.

An activation button 120 is disposed on housing 112 and coupled to or between ultrasonic transducer 140 and/or surgical generator 200, e.g., via one or more of first electrical lead wires 197, to enable activation of ultrasonic transducer 140 in response to depression of activation button 120. In some configurations, activation button 120 may include an ON/OFF switch. In other configurations, activation button 120 may include multiple actuation switches to enable activation from an OFF position to different actuated positions corresponding to different activation settings, e.g., a first actuated position corresponding to a first activation setting (such as a LOW power or tissue sealing setting) and a second actuated position corresponding to a second activation setting (such as a HIGH power or tissue transection setting). In still other configurations, separate activation buttons may be provided, e.g., a first actuation button for activating a first activation setting and a second activation button for activating a second activation setting. Additional activation buttons, sliders, wheels, etc. are also contemplated to enable control of various different activation settings from housing 112.

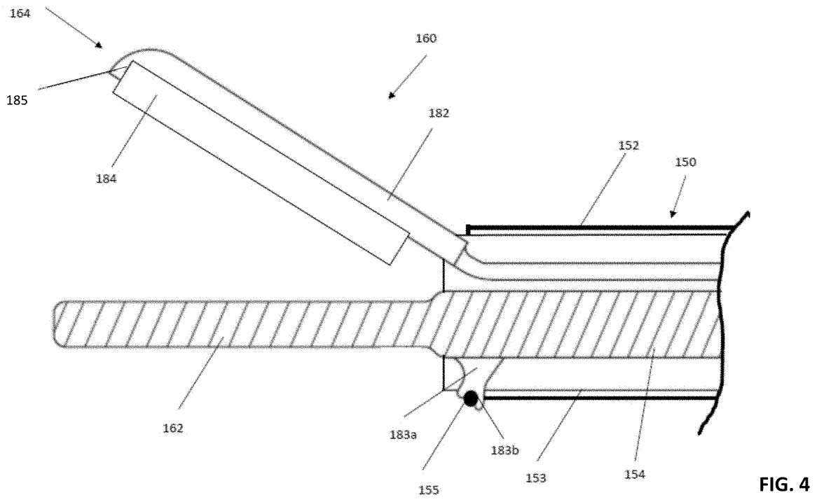

Elongated assembly 150 of surgical instrument 100 includes an outer drive sleeve 152, an inner support sleeve 153 (FIG. 4) disposed within outer drive sleeve 152, a waveguide 154 extending through inner support sleeve 153 (FIG. 4), a drive assembly (not shown), a rotation knob 156, and an end effector assembly 160 including a blade 162 and a jaw member 164. In aspects where only electrosurgical energy is provided and/or other configurations, waveguide 154 and blade 162 may be replaced with a second jaw member (not shown) configured to oppose jaw member 164. Rotation knob 156 is rotatable in either direction to rotate elongated assembly 150 in either direction relative to handle assembly 110. The drive assembly operably couples a proximal portion of outer drive sleeve 152 to clamp trigger 130 of handle assembly 110. A distal portion of outer drive sleeve 152 is operably coupled to jaw member 164 and a distal end of inner support sleeve 153 (FIG. 4) pivotably supports jaw member 164. As such, clamp trigger 130 is selectively actuatable to thereby move outer drive sleeve 152 about inner support sleeve 153 (FIG. 4) to pivot jaw member 164 relative to blade 162 of end effector assembly 160 from a spaced apart position to an approximated position for clamping tissue between jaw member 164 and blade 162. The configuration of outer and inner sleeves 152, 153 (FIG. 4) may be reversed, e.g., wherein outer sleeve 152 is the support sleeve and inner sleeve 153 (FIG. 4) is the drive sleeve. Other suitable drive structures as opposed to a sleeve are also contemplated such as, for example, drive rods, drive cables, drive screws, etc.

Referring still to FIG. 1, the drive assembly may be tuned to provide a jaw clamping force, or jaw clamping force within a jaw clamping force range, to tissue clamped between jaw member 164 and blade 162 or may include a force limiting feature whereby the clamping force applied to tissue clamped between jaw member 164 and blade 162 is limited to a particular jaw clamping force or a jaw clamping force within a jaw clamping force range.

Waveguide 154, as noted above, extends from handle assembly 110 through inner sleeve 153 (FIG. 4). Waveguide 154 includes blade 162 disposed at a distal end thereof. Blade 162 may be integrally formed with waveguide 154, separately formed and subsequently attached (permanently or removably) to waveguide 154, or otherwise operably coupled with waveguide 154. Waveguide 154 and/or blade 162 may be formed from titanium, a titanium alloy, or other suitable electrically conductive material(s), although non-conductive materials are also contemplated. Waveguide 154 includes a proximal connector (not shown), e.g., a threaded male connector, configured for engagement, e.g., threaded engagement within a threaded female receiver, of ultrasonic transducer 140 such that ultrasonic motion produced by ultrasonic transducer 140 is transmitted along waveguide 154 to blade 162 for treating tissue clamped between blade 162 and jaw member 164 or positioned adjacent to blade 162.

Cable assembly 190 of surgical instrument 100 includes a cable 192, an ultrasonic plug 194, and an electrosurgical plug 196. Ultrasonic plug 194 is configured for connection with ultrasonic plug port 230 of surgical generator 200 while electrosurgical plug 196 is configured for connection with bipolar electrosurgical plug port 240 of surgical generator 200 and/or active monopolar electrosurgical plug port 250 of surgical generator 200. In configurations where generator 200 includes a common port, cable assembly 190 may include a common plug (not shown) configured to act as both the ultrasonic plug 194 and the electrosurgical plug 196. In configurations where surgical instrument 100 is only configured for ultrasonic operation, electrosurgical plug 196 and associated components are omitted.

Plural first electrical lead wires 197 electrically coupled to ultrasonic plug 194 extend through cable 192 and into handle assembly 110 for electrical connection to ultrasonic transducer 140 and/or activation button 120 to enable the selective supply of ultrasonic drive signals from surgical generator 200 to ultrasonic transducer 140 upon activation of activation button 120 in an ultrasonic mode. In addition, plural second electrical lead wires 199 are electrically coupled to electrosurgical plug 196 and extend through cable 192 into handle assembly 110. In bipolar configurations, separate second electrical lead wires 199 are electrically coupled to waveguide 154 and jaw member 164 (and/or different portions of jaw member 164) such that bipolar electrosurgical energy may be conducted between blade 162 and jaw member 164 (and/or between different portions of jaw member 164). In monopolar configurations, a second electrical lead wire 199 is electrically coupled to waveguide 154 such that monopolar electrosurgical energy may be supplied to tissue from blade 162. Alternatively or additionally, a second electrical lead wire 199 may electrically couple to jaw member 164 in the monopolar configuration to enable monopolar electrosurgical energy to be supplied to tissue from jaw member 164. In configurations where both bipolar and monopolar functionality are enabled, one or more of the second electrical lead wires 199 may be used for both the delivery of bipolar energy and monopolar energy; alternatively, bipolar and monopolar energy delivery may be provided by separate second electrical lead wires 199. One or more other second electrical lead wires 199 is electrically coupled to activation button 120 to enable the selective supply of electrosurgical energy from surgical generator 200 to waveguide 154 and/or jaw member 164 upon activation of activation button 120 in an electrosurgical mode(s).

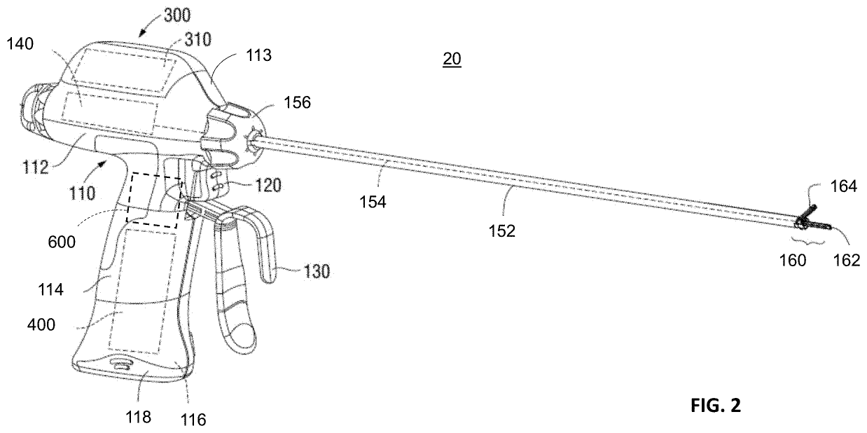

As an alternative to a remote generator 200, surgical system 10 may be at least partially cordless in that it incorporates an ultrasonic generator, an electrosurgical generator, and/or a power source, e.g., a battery, thereon or therein. In this manner, the connections from surgical instrument 100 to external devices, e.g., generator(s) and/or power source(s), is reduced or eliminated. More specifically, with reference to FIG. 2, another surgical system in accordance with the present disclosure is shown illustrated as a surgical instrument 20 supporting an ultrasonic generator 310, a power source (e.g., battery assembly 400), and an electrosurgical generator 600 thereon or therein. Surgical instrument 20 is similar to surgical instrument 100 (FIG. 1) and may include any of the features thereof except as explicitly contradicted below. Accordingly, only differences between surgical instrument 20 and surgical instrument 100 (FIG. 1) are described in detail below while similarities are omitted or summarily described.

Housing 112 of surgical instrument 20 includes a body portion 113 and a fixed handle portion 114 depending from body portion 113. Body portion 113 of housing 112 is configured to support an ultrasonic transducer and generator assembly (“TAG”) 300 including ultrasonic generator 310 and ultrasonic transducer 140. TAG 300 may be permanently engaged with body portion 113 of housing 112 or removable therefrom.

Fixed handle portion 114 of housing 112 defines a compartment 116 configured to receive battery assembly 400 and electrosurgical generator 600 and a door 118 configured to enclose compartment 116. An electrical connection assembly (not shown) is disposed within housing 112 and serves to electrically couple activation button 120, ultrasonic generator 310 of TAG 300, and battery assembly 400 with one another when TAG 300 is supported on or in body portion 113 of housing 112 and battery assembly 400 is disposed within compartment 116 of fixed handle portion 114 of housing 112, thus enabling activation of surgical instrument 20 in an ultrasonic mode in response to appropriate actuation of activation button 120. Further, the electrical connection assembly or a different electrical connection assembly disposed within housing 112 serves to electrically couple activation button 120, electrosurgical generator 600, battery assembly 400, and end effector assembly 160 (e.g., blade 162 and jaw member 164 and/or different portions of jaw member 164) with one another when electrosurgical generator 600 and battery assembly 400 are disposed within compartment 116 of fixed handle portion 114 of housing 112, thus enabling activation of surgical instrument 20 in an electrosurgical mode, e.g., bipolar RF, in response to appropriate actuation of activation button 120. For a monopolar electrosurgical mode, return electrode device 500 (FIG. 1) may be configured to connect to surgical instrument 20 (electrosurgical generator 600 thereof, more specifically), to complete a monopolar circuit through tissue and between surgical instrument 20 (e.g., blade 162 and/or jaw member 164) and return electrode device 500 (FIG. 1).

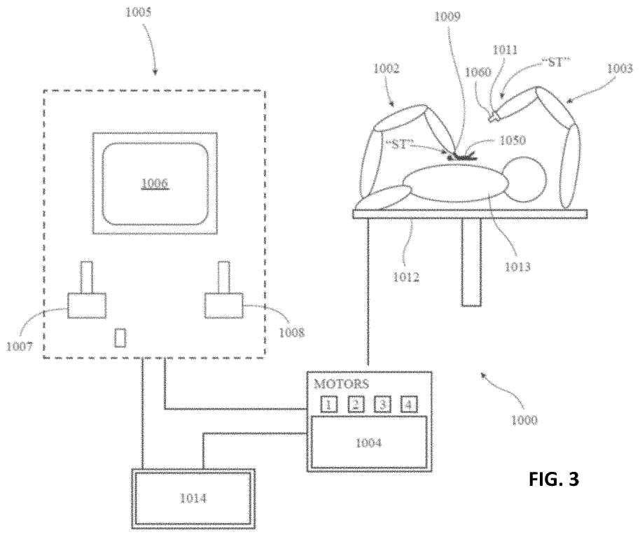

Turning to FIG. 3, a robotic surgical system in accordance with the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three dimensional images; and manual input devices 1007, 1008, by means of which a person (not shown), for example a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, a surgical tool “ST” supporting an end effector 1050, 1060. One of the surgical tools “ST” may be surgical instrument 100 (FIG. 1), surgical instrument 20 (FIG. 2), or any other suitable surgical instrument 20 configured for use in both an ultrasonic mode and one or more electrosurgical (bipolar and/or monopolar) modes, wherein manual actuation features, e.g., actuation button 120 (FIG. 1), clamp lever 130 (FIG. 1), etc., are replaced with robotic inputs. In such configurations, robotic surgical system 1000 may include or be configured to connect to an ultrasonic generator, an electrosurgical generator, and/or a power source. The other surgical tool “ST” may include any other suitable surgical instrument, e.g., an endoscopic camera, other surgical tool, etc. Robot arms 1002, 1003 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and, thus, the surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

Referring to FIGS. 4-6, end effector assembly 160 of surgical instrument 100 of surgical system 10 (FIG. 1) is detailed, although the aspects and features of end effector assembly 160 may similarly apply, to the extent consistent, to surgical instrument 20 (FIG. 2) and/or any other suitable surgical instrument or system. End effector assembly 160, as noted above, includes blade 162 and jaw member 164. Blade 162 may define a linear configuration, may define a curved configuration, or may define any other suitable configuration, e.g., straight and/or curved surfaces, portions, and/or sections; one or more convex and/or concave surfaces, portions, and/or sections; etc. With respect to curved configurations, blade 162, more specifically, may be curved in any direction relative to jaw member 164, for example, such that the distal tip of blade 162 is curved towards jaw member 164, away from jaw member 164, or laterally (in either direction) relative to jaw member 164. Further, blade 162 may be formed to include multiple curves in similar directions, multiple curves in different directions within a single plane, and/or multiple curves in different directions in different planes. In addition, blade 162 may additionally or alternatively be formed to include any suitable features, e.g., a tapered configuration, various different cross-sectional configurations along its length, cut outs, indents, edges, protrusions, straight surfaces, curved surfaces, angled surfaces, wide edges, narrow edges, and/or other features.

Blade 162 may define a polygonal, rounded polygonal, or any other suitable cross-sectional configuration(s). Waveguide 154 or at least the portion of waveguide 154 proximally adjacent blade 162, may define a cylindrical shaped configuration. Plural tapered surfaces (not shown) may interconnect the cylindrically shaped waveguide 154 with the polygonal (rounded edge polygonal, or other suitable shape) configuration of blade 162 to define smooth transitions between the body of waveguide 154 and blade 162.

Blade 162 may be wholly or selectively coated with a suitable material, e.g., a non-stick material, an electrically insulative material, an electrically conductive material, combinations thereof, etc. Suitable coatings and/or methods of applying coatings include but are not limited to Teflon®, polyphenylene oxide (PPO), deposited liquid ceramic insulative coatings; thermally sprayed coatings, e.g., thermally sprayed ceramic; Plasma Electrolytic Oxidation (PEO) coatings; anodization coatings; sputtered coatings, e.g., silica; ElectroBond® coating available from Surface Solutions Group of Chicago, Ill., USA; or other suitable coatings and/or methods of applying coatings.

Continuing with reference to FIGS. 4-6, blade 162, as noted above, in addition to receiving ultrasonic energy transmitted along waveguide 154 from ultrasonic transducer 140 (FIG. 1), may be adapted to connect to generator 200 (FIG. 1) to enable the supply of RF energy to blade 162 for conduction to tissue in contact therewith. In bipolar configurations, RF energy is conducted between blade 162 and jaw member 164 (or between portions of jaw member 164 and/or blade 162) and through tissue disposed therebetween to treat tissue. In monopolar configurations, RF energy is conducted from blade 162, serving as the active electrode, to tissue in contact therewith and is ultimately returned to generator 200 (FIG. 1) via return electrode device 500 (FIG. 1), serving as the passive or return electrode.

Jaw member 164 of end effector assembly 160 includes more rigid structural body 182 and more compliant jaw liner 184. Structural body 182 may be formed from an electrically conductive material, e.g., stainless steel, and/or may include electrically conductive portions. Structural body 182 includes a pair of proximal flanges 183a that are pivotably coupled to the inner support sleeve 153 via receipt of pivot bosses (not shown) of proximal flanges 183a within corresponding openings (not shown) defined within the inner support sleeve 153 and operably coupled with outer drive sleeve 152 via a drive pin 155 secured relative to outer drive sleeve 152 and pivotably received within apertures 183b defined within proximal flanges 183a. As such, sliding of outer drive sleeve 152 about inner support sleeve 153 pivots jaw member 164 relative to blade 162 from a spaced apart position to an approximated position to clamp tissue between jaw liner 184 of jaw member 164 and blade 162.

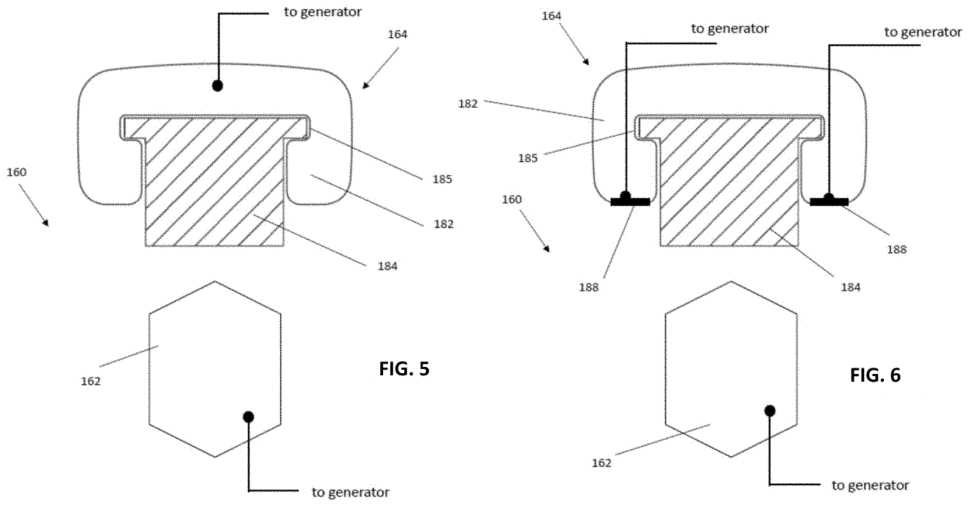

With reference to FIG. 5, structural body 182 may be adapted to connect to a source of electrosurgical energy, e.g., generator 200 (FIG. 1), and, in a bipolar electrosurgical mode, is charged to a different potential as compared to blade 162 to enable the conduction of bipolar electrosurgical (e.g., RF) energy through tissue clamped therebetween, to treat the tissue. In a monopolar electrosurgical mode, structural body 182 may be un-energized, may be charged to the same potential as compared to blade 162 (thus both defining the active electrode), or may be energized while blade 162 is not energized (wherein structural body 182 defines the active electrode). In either monopolar configuration, energy is returned to generator 200 (FIG. 1) via return electrode device 500 (FIG. 1), which serves as the passive or return electrode.

Referring to FIG. 6, as an alternative to the entirety of structural body 182 of jaw member 164 being connected to generator 200 (FIG. 1), the structural body may be formed from or embedded at least partially in an insulative material, e.g., an overmolded plastic, or a conductive material coated or otherwise treated to be non-conductive, e.g., PEO-coated titanium, ceramic-coating steel, etc. In such configurations, electrically conductive surfaces 188, e.g., in the form of plates, may be disposed on (e.g., bonded to, deposited onto, mechanically engaged with, etc.) or captured by the insulative material (e.g., overmolded plastic) to define electrodes on either side of jaw liner 184 on the blade facing side of jaw member 164. The electrically conductive surfaces 188, in such aspects, are connected to generator 200 (FIG. 1) and may be energized for use in bipolar and/or monopolar configurations, e.g., energized to the same potential as one another and/or blade 162 and/or different potentials as one another and/or blade 162. In aspects, electrically conductive surfaces 188 are disposed at additional or alternative locations on jaw member 164, along either or both sides thereof, along a back surface thereof, etc.

Again referring to FIGS. 4-6, jaw liner 184 is retained within a cavity 185 defined within structural body 182. Jaw liner 184 is fabricated from an electrically insulative, compliant material such as, for example, polytetrafluoroethylene (PTFE), although other suitable materials, including conductive materials, partially conductive and partially non-conductive materials, Positive Temperature Coefficient (PTC) materials, Negative Temperature Coefficient (NTC) materials, combinations of the above and/or other materials, etc. are also contemplated. The compliance of jaw liner 184 enables blade 162 to vibrate while in contact with jaw liner 184 without damaging components of ultrasonic surgical instrument 100 (FIG. 1) and without compromising the hold on tissue clamped between jaw member 164 and blade 162. As an alternative to jaw member 164 and, more specifically, jaw liner 184 thereof opposing blade 162 in the approximated position of jaw member 164, jaw member 164 (including jaw liner 184) may be utilized in an end effector assembly having an opposing jaw member (not shown), e.g., for transmission of energy (electrosurgical, thermal, microwave, light, ultrasonic, etc.) to tissue to seal and/or cut tissue grasped therebetween. In such configurations, the opposing jaw member (not shown) may include a thermal cutting element configured to thermally cut tissue and positioned to oppose jaw liner 184 (wherein jaw liner 184 functions as the insulative member of the jaw member), for example, as described in Patent Application Pub. US 2021/0186587, the entire contents of which are hereby incorporated herein by reference.

Jaw liner 184, in aspects, extends from structural body 182 towards blade 162 to inhibit contact between structural body 182 and blade 162 in the approximated position of jaw member 164. The insulation of jaw liner 184 maintains electrical isolation between blade 162 and structural body 182 of jaw member 164, thereby inhibiting shorting.

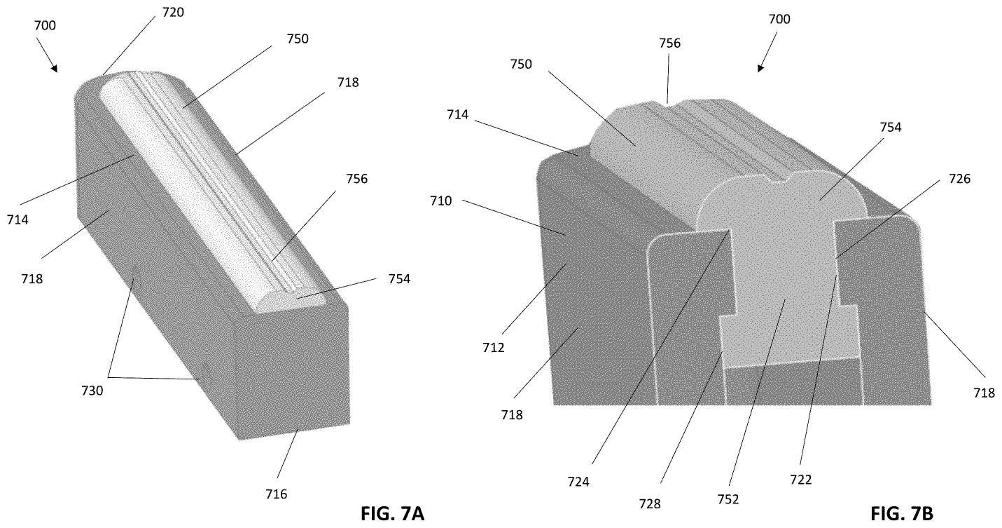

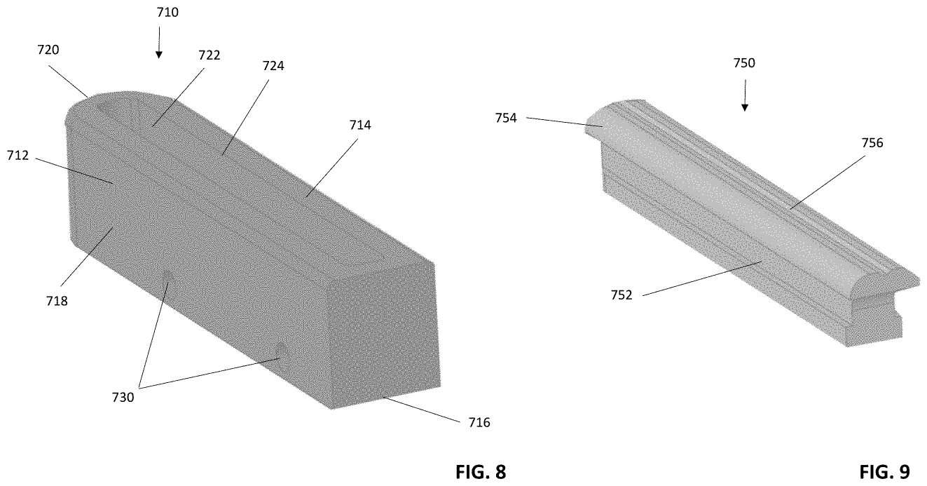

Turning to FIGS. 7A-9, a portion of a jaw member 700 provided in accordance with the present disclosure is shown. Jaw member 700 may be similar to and include any of the features (or combination of features) of the various configurations of jaw member 164 (FIG. 4) detailed hereinabove. Alternatively, jaw member 700 may be configured similar to any other suitable jaw member having a structural body and a jaw liner (also referred to in the art, instead of a liner, as a pad, insulator, insert, spacer, or other like structure associated with a structural body of a jaw member). Thus, jaw member 700 may be utilized with surgical instrument 100 (FIG. 1), surgical instrument 20 (FIG. 2), or any other suitable surgical instrument. Jaw member 700 includes a structural body 710 and a jaw liner 750.

Structural body 710 of jaw member 700 may be formed from any suitable material, e.g., stainless steel, and may be machined or formed in any other suitable manner. Structural body 710 and/or one or more components disposed thereon may be connected to a source of electrical energy (electrical, thermal, microwave, light) to deliver energy to tissue or may only be used to facilitate grasping and manipulating tissue. Structural body 710 includes a base 712 defining a tissue-facing surface 714, a back surface 716, a pair of side walls 718, a distal end 720, and a proximal end (not shown, including, in aspects, one or more proximal flanges 183a (FIG. 4) or other suitable connecting structures for pivotably, otherwise movably, or fixedly coupling structural body 710 of jaw member 700 to a support and/or actuator). Structural body 710 further includes an internal cavity 722 defined therein and communicating with an elongated opening 724 defined through tissue-facing surface 714. Base 712 of structural body 710 may otherwise enclose internal cavity 722, e.g., such that internal cavity is not accessible via back surface 716, side walls 718, distal end 720, or the proximal end, although other configurations are also contemplated.

Internal cavity 722 of structural body 710 defines a non-uniform shape such as, for example, including a first portion 726 defining a first width and a second portion 728 defining a second width different from the first width. The first and second widths may be consistent or may vary (similarly or differently) along the lengths of first and second portions 726, 728. The widths and/or lengths of first and second portions 726, 728 may be similar or different. In aspects, the first width of first portion 726 is smaller than the second width of second portion 728 along at least portions of lengths thereof and first portion 726 is disposed in direct communication with elongated opening 724 while the second portion 728 is disposed in communication with elongated opening 724 via first portion 726. That is, internal cavity 722 may define an upside down T-shaped configuration (as viewed from the orientation shown in FIG. 7B) along at least a portion of a length thereof. The first and second portions 726, 728 may define similar or different heights that may be consistent or vary along the length of internal cavity 722. Other polygonal (including vertical or angled walls), curved, or combinations of polygonal and curved configurations of first and second portions 726, 728 are also contemplated, as are greater or fewer than two portions defining internal cavity 722. Regardless of the particular shape of internal cavity 722 (or the portions thereof), internal cavity 722 is configured to retain a portion of jaw liner 750 therein such that, once jaw liner 750 is formed at least partially within internal cavity 722, the portion of jaw liner 750 is retained therein in substantially fixed relation relative to structural body 710. This may be accomplished via providing the first width of first portion 726 smaller than the second width of second portion 728 along at least portions of lengths thereof as detailed above, or in any other suitable manner.

Elongated opening 724 and/or internal cavity 722 may be laterally centered relative to tissue-facing surface 714 of base 712 of structural body 710 or may be laterally offset relative thereto. Further, elongated opening 724 and/or internal cavity 722 may extend from the proximal end of tissue-facing surface 714 to a position proximally-spaced from the distal end thereof, may extend from a position proximally-spaced from the proximal end of tissue-facing surface 714 to the distal end of tissue-facing surface 714, or may be spaced-apart from both the proximal and distal ends of tissue-facing surface 714. Elongated opening 714 may additionally or alternatively define a curvature and one or more angled segments, similarly as a curvature or angle of base 712 of structural body 710 or differently therefrom. Further still, internal cavity 722 may be symmetric or asymmetric with respect to elongated opening 724.

In aspects, structural body 710 includes one or more apertures 730 and/or other features (e.g., protrusions, slots, etc.) defined therein, e.g., through either or both sidewalls 718 thereof, to facilitate retention of structural body 710 in a fixture (not shown), e.g., to facilitate formation of jaw liner 750 at least partially within internal cavity 722 of base 712 of structural body 710.

Continuing with reference to FIGS. 7A-9, jaw liner 750 is configured for receipt at least partially within internal cavity 722 of base 712 of structural body 710 and extends to or through elongated opening 724 thereof. Jaw liner 750 may be formed from an insulative material, conductive material, or partially conductive and partially non-conductive material. Further, jaw line 750, in aspects, may be formed form a compliant material. As examples, jaw liner 750 may be formed from polytetrafluoroethylene (PTFE), Positive Temperature Coefficient (PTC) materials, Negative Temperature Coefficient (NTC) materials, combinations of the above and/or other materials, etc. While materials that flow when heated above their melting point in a manner that enables injection molding and/or overmolding are contemplated, materials that do not flow in this manner or are otherwise not conducive to injection molding and/or overmolding, such as PTFE, are particularly suitable for use in forming jaw liner 750 in accordance with the aspects and features of the present disclosure.

Jaw liner 750 is not pre-formed and inserted into structural body 710 without or with minimal manipulation thereof but, rather, is formed to its final configuration and secured within structural body 710 via forging. Thus, jaw liner 750 as shown in FIG. 9 is provided for illustration purposes only as jaw liner 750 would not assume this configuration in the absence of and until it is forged into structural body 710. Forging is advantageous at least in that it may be used on materials that do not retain desired material properties after being heated above their melting points, materials where heating the material above its melting point is not desired (e.g., where certain gasses may be released), and/or materials that are otherwise not conducive to injection molding or overmolding, although it can also be used on moldable materials. In aspects, jaw liner 750 is formed to its final configuration and secured within structural body 710 via die forging (e.g., via open die forging or closed die forging (also referred to as impression die forging)). In this manner, the material 752 of jaw liner 750 is forged through elongated opening 724 and into internal cavity 722 in a more-malleable state (as a result of the heat and/or pressure applied thereto) that enables the material to enter and substantially conform to the configuration of internal cavity 722 and, once set (i.e., allowed to cool or otherwise return to the less-malleable, final form thereof), is fixedly secured therein. For example, as shown in FIG. 7B, the material 752 of jaw liner 750 that is forged through elongated opening 724 and into internal cavity 722 may define an upside down T-shaped configuration along at least a portion of a length thereof, complementary to the shape of internal cavity 722.

The material 754, if any, of jaw liner 750 that remains external of internal cavity 722 may be forged to define any suitable configuration extending from elongated opening 724 and/or tissue-facing surface 714 of base 712 of structural body 710. For example, the material 754 of jaw liner 750 that remains external of internal cavity 722 may extend outwardly from elongated opening 724 onto portions of tissue-facing surface 714 on either side of elongated opening 724 (and onto a portion of tissue-facing surface 714 disposed distally of elongated opening 724) so as to define an apron surrounding elongated opening 724. Additionally, or alternatively, the material 754 may protrude from tissue-facing surface 714 elongated opening 724 and define a semi-circular transverse cross-sectional configuration (as shown) or any other suitable transverse cross-sectional configuration. One or more features 756 may be formed on or within the material 754 such as, for example, one or more longitudinal channels (as shown), one or more longitudinal protrusions, one or more transverse channels and/or protrusions, grasping teeth, grasping recesses, etc. The one or more features 756 may additionally or alternatively include surface features such as, for example, saw-tooth, sine-wave, stepped, and/or other suitable surface features formed on the material 754. The particular configuration of the material 754 and/or features 756 thereof may be established by use of appropriate forging tools (e.g., wherein the forging die(s) are configured to achieve a desired configuration and/or feature(s) of the material 754).

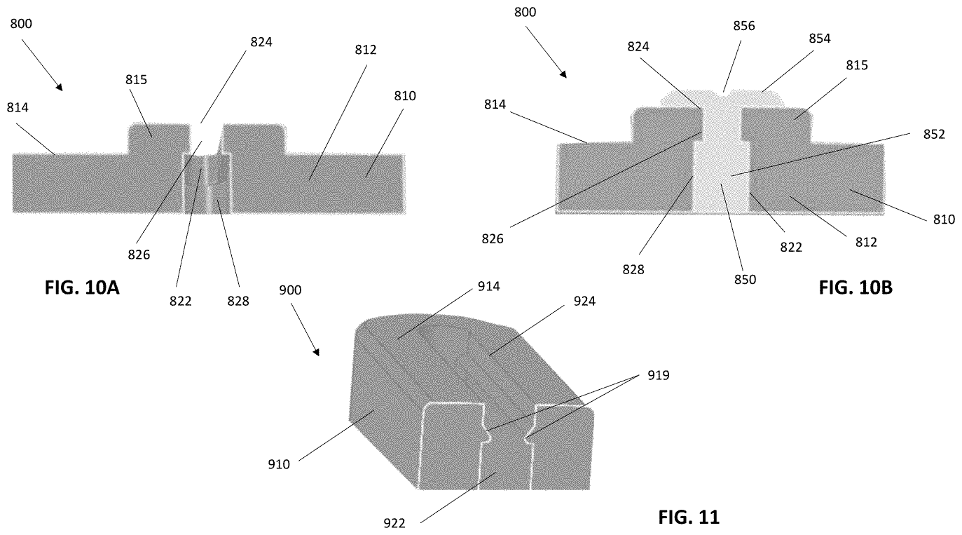

Referring to FIGS. 10A and 10B, another portion of a jaw member 800 provided in accordance with the present disclosure is shown. Jaw member 800 may be similar to and include any of the features of jaw member 700 (FIGS. 7A-9) and, thus, only differences therebetween are described in detail hereinbelow. Jaw member 800 includes a structural body 810 and a jaw liner 850.

Structural body 810 of jaw member 800 includes a raised mesa 815 extending from tissue-facing surface 814. Structural body 810 further includes an internal cavity 822 defined therein and communicating with an elongated opening 824 defined through raised mesa 815. Elongated opening 824 and/or raised mesa 815 may be laterally centered on tissue-facing surface 814 or offset relative thereto. Internal cavity 822 of structural body 810 includes a first portion 826 (corresponding to the portion defined through raised mesa 815) in direct communication with elongated opening 824 and defining a first width and height, and a second portion 828 (corresponding to the portion defined through base 812) in indirect communication with elongated opening 824 and defining a second width and height. The second width and height are greater than the first width and height, respectively, although other configurations are also contemplated. In aspects, as an alternative or in addition to a raised mesa 815, structural body 810 may include one or more other features defined on or within tissue-facing surface 814 such as, for example, continuous and/or discrete protrusions and/or steps that are rounded, angled, etc.

Jaw liner 850 is formed to its final configuration and secured within structural body 810 via forging. The material 852 of jaw liner 850 that is forged through elongated opening 824 and into internal cavity 822 defines a configuration complementary to the shape of internal cavity 822. The material 854 of jaw liner 850 that remains external of internal cavity 822 is forged to extend outwardly on either side of elongated opening 824 to cover at least a portion of the substantially flat tissue-facing surface of raised mesa 815 and defines a substantially planar raised surface with rounded edges extending along the length thereof (and a rounded distal end, in aspects). Further, a feature 856, e.g., an elongated channel, is defined within the substantially planar raised surface of the material 854. The channel may be configured to receive an apex of an opposing structure, e.g., of an ultrasonic blade or of a thermal cutting element of an opposing jaw member, when jaw member 800 is disposed in the approximated position for grasping tissue in conjunction with the opposing structure.

With reference to FIG. 11, another structural body 910 configured for use with any of the jaw members (or jaw liners) detailed herein or any other suitable jaw member (or jaw liner) is provided in accordance with the present disclosure is shown. Structural body 910 may be similar to and include any of the features of structural bodies 710, 810 (FIGS. 7A-8 and FIGS. 10A-10B, respectively) and, thus, only differences therebetween are described in detail hereinbelow.

Structural body 910 of jaw member 900 includes an internal cavity 922 defined therein and communicating with an elongated opening 924 defined through tissue-facing surface 914 thereof. Internal cavity 922 of structural body 910 defines a substantially rectangular transverse cross-sectional configuration. Structural body 910 includes a pair of retention features 919 that protrude inwardly into internal cavity 922 from opposing sidewalls thereof. Retention features 919 may be elongated ribs defining triangular transverse cross-sectional configurations, or may be any other suitable retention features, e.g., elongated or discrete protruding structures on either or both sides of internal cavity 922 along the length thereof, plural retention features along the height of internal cavity 922, shelves, angled surfaces, etc. Regardless of the particular configuration, retention features 919 facilitate retention of a jaw liner within structural body 910 when the jaw liner is forged and set within internal cavity 922.

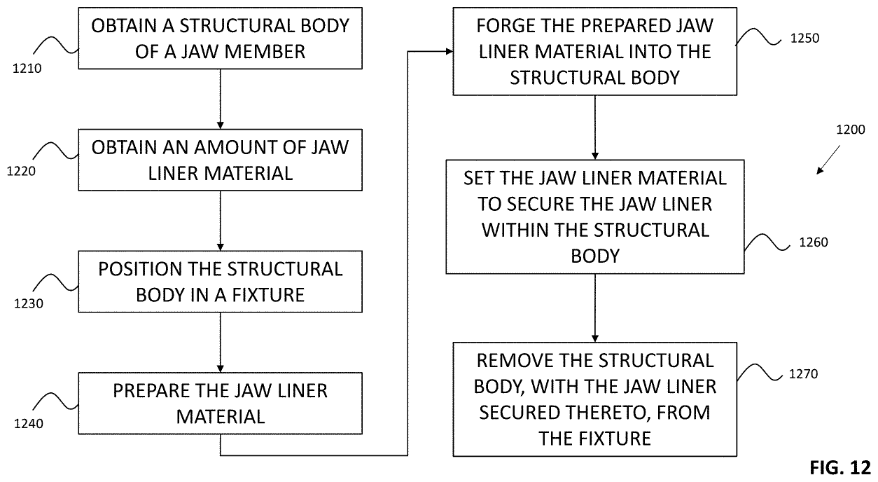

Turning to FIG. 12, a method of manufacturing a jaw member in accordance with the present disclosure is illustrated and identified as method 1200. At steps 1210 and 1220, a structural body of a jaw member and an amount of jaw liner material, respectively, are obtained. The structural body of the jaw member is positioned in a fixture at step 1230 such as, for example, within a die fixture for die forging. At step 1240, the jaw liner material is prepared such as, for example, heated to a suitable temperature and/or pressurized for forging, or otherwise prepared to achieve a more-malleable state that enables forging. Thereafter, at step 1250, the jaw liner material is forged into the structural body such that at least a portion of the jaw liner material substantially fills and conforms to the shape of a cavity defined within the structural body and, in aspects, such that a portion of the jaw liner material extends from the structural body to define a suitable configuration and/or features. This may be accomplished by relative movement between one or more dies and the die fixture, or in any other suitable manner. At step 1260 the jaw liner material is set to its final form, thereby securing the jaw liner within the structural body. Setting the jaw liner material may be accomplished by allowing the heated jaw liner material to cool, by actively cooling the heated jaw liner material, by releasing or removing the forge pressure, or setting the jaw liner material in any other suitable manner to retain its final form. Finally, at step 1270, the structural body, including the jaw liner secured thereto, is removed from the fixture.

While several aspects of the disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular aspects. Those skilled in the art will envision other modifications within the scope and spirit of the claims appended hereto.

What is claimed is:

1. A jaw member of a surgical instrument, comprising:
a structural body including a tissue-facing surface having an elongated opening defined therethrough, the structural body defining an internal cavity in communication with the elongated opening; and
a jaw liner of a material that is not conducive to molding, the jaw liner including an internal portion substantially filling the internal cavity and extending to the elongated opening.

2. The jaw member according to claim 1, wherein the material is PTFE.

3. The jaw member according to claim 1, wherein the jaw liner includes an external portion that extends from the elongated opening and at least one of protrudes from the tissue-facing surface or extends outwardly over the tissue-facing surface.

4. The jaw member according to claim 3, wherein the external portion of the jaw liner protrudes from the tissue-facing surface and extends outwardly over the tissue-facing surface.

5. The jaw member according to claim 3, wherein the external portion of the jaw liner defines at least one feature therein or thereon.

6. The jaw member according to claim 1, wherein the internal cavity defines first and second portions having different widths, the first portion disposed in direct communication with the elongated opening and the second portion communicating with the elongated opening through the first portion.

7. The jaw member according to claim 1, wherein the structural body includes at least one retention feature extending into the internal cavity and configured to facilitate retention of the jaw liner.

8. The jaw member according to claim 1, wherein the structural body includes a raised mesa extending from the tissue-facing surface, wherein the elongated opening is defined through the raised mesa.

9. The jaw member according to claim 8, wherein the jaw liner includes an external portion that extends from the elongated opening outwardly over at least a portion of the raised mesa.

10. The jaw member according to claim 1, wherein, except for the elongated opening, the structural body substantially encloses the internal cavity.

11. A method of manufacturing a jaw member of a surgical instrument, the method comprising:
preparing a material;
forging the material into an internal cavity defined within a structural body of a jaw member; and
setting the material such that the material substantially fills the internal cavity and is retained therein thereby forming a jaw liner of the jaw member.

12. The method according to claim 11, further comprising, prior to forging the material, positioning the structural body within a fixture.

13. The method according to claim 11, wherein the forging includes impression die forging.

14. The method according to claim 11, wherein preparing the material includes heating the material.

15. The method according to claim 11, wherein the material is PTFE.

16. The method according to claim 11, wherein the material is not conducive to molding.

17. The method according to claim 11, wherein forging the prepared material includes urging the material through an elongated opening defined within a tissue-facing surface of the structural body and into the internal cavity.

18. The method according to claim 11, wherein setting the material includes actively cooling the material or allowing the material to cool.

19. The method according to claim 11, wherein forging the material into the internal cavity defined within the structural body further includes forging a portion of the material externally of the structural body.

20. The method according to claim 19, wherein forging the portion of the material externally of the structural body includes protruding the portion of material from or extending the portion of material along the tissue-facing surface of the structural body.
